SEQUENCE LISTING (26 sequences)

SEQ ID NO: 1 - protein, 7 aa, synthetic construct
PKKKRKV

SEQ ID NO: 2 - protein, 16 aa, synthetic construct
LLLLLLLLLPKKKRKV

SEQ ID NO: 3 - protein, 23 aa, synthetic construct
LLLLLLLLLPKKKRKVPKKKRKV

SEQ ID NO: 4 - protein, 30 aa, synthetic construct
LLLLLLLLLPKKKRKVPKKKRKVPKKKRKV

SEQ ID NO: 5 - protein, 37 aa, synthetic construct
LLLLLLLLLPKKKRKVPKKKRKVPKKKRKVPKKKRKV

SEQ ID NO: 6 - protein, 17 aa, synthetic construct
LLLLLLLLLLPKKKRKV

SEQ ID NO: 7 - protein, 24 aa, synthetic construct
LLLLLLLLLLPKKKRKVPKKKRKV

SEQ ID NO: 8 - protein, 31 aa, synthetic construct
LLLLLLLLLLPKKKRKVPKKKRKVPKKKRKV

SEQ ID NO: 9 - protein, 38 aa, synthetic construct
LLLLLLLLLLPKKKRKVPKKKRKVPKKKRKVPKKKRKV

SEQ ID NO: 10 - protein, 18 aa, synthetic construct
LLLLLLLLLLLPKKKRKV

SEQ ID NO: 11 - protein, 25 aa, synthetic construct
LLLLLLLLLLLPKKKRKVPKKKRKV

SEQ ID NO: 12 - protein, 32 aa, synthetic construct
LLLLLLLLLLLPKKKRKVPKKKRKVPKKKRKV

SEQ ID NO: 13 - protein, 39 aa, synthetic construct
LLLLLLLLLLLPKKKRKVPKKKRKVPKKKRKVPKKKRKV

SEQ ID NO: 14 - protein, 9 aa, synthetic construct
LLLLLLLLL

SEQ ID NO: 15 - protein, 10 aa, synthetic construct
LLLLLLLLLL

SEQ ID NO: 16 - protein, 11 aa, synthetic construct
LLLLLLLLLLL

SEQ ID NO: 17 - protein, 24 aa, synthetic construct
FFFFFFFFFFPKKKRKVPKKKRKV

SEQ ID NO: 18 - protein, 24 aa, synthetic construct
MMMMMMMMMMPKKKRKVPKKKRKV

SEQ ID NO: 19 - protein, 24 aa, synthetic construct
VVVVVVVVVVPKKKRKVPKKKRKV

SEQ ID NO: 20 - protein, 24 aa, synthetic construct
GGGGGGGGGGPKKKRKVPKKKRKV

SEQ ID NO: 21 - protein, 24 aa, synthetic construct
PPPPPPPPPPPKKKRKVPKKKRKV

SEQ ID NO: 22 - protein, 24 aa, synthetic construct
AAAAAAAAAAPKKKRKVPKKKRKV

SEQ ID NO: 23 - protein, 24 aa, synthetic construct
YYYYYYYYYYPKKKRKVPKKKRKV

SEQ ID NO: 24 - protein, 24 aa, synthetic construct
IIIILIIIILPKKKRKVPKKKRKV

SEQ ID NO: 25 - protein, 24 aa, synthetic construct
WWWWLWWWWLPKKKRKVPKKKRKV

SEQ ID NO: 26 - protein, 24 aa, synthetic construct
PKKKRKVPKKKRKVLLLLLLLLLL

(19) United States
(12) Patent Application Publication
(10) Pub. No.: US 2025/0368682 A1
(43) Pub. Date: Dec. 4, 2025

(54) NOVEL POLYPEPTIDE COMPOSITION FOR INTRACELLULAR TRANSFECTION

(71) Applicant: AZOTHBIO INC., Gyeonggi-do (KR)

(72) Inventors: Jae Min Shin, Gyeonggi-do (KR); Jung-Hyun Park, Seoul (KR); Ji Yeon Chang, Gyeonggi-do (KR); Song Mi Kim, Gyeonggi-do (KR); Meriem Bouchane, Seoul (KR)

(21) Appl. No.: 19/108,151

(22) PCT Filed: Sep. 7, 2023
(86) PCT No.: PCT/KR2023/013425

§ 371 Date: Feb. 28, 2025

(30) Foreign Application Priority Data
Sep. 8, 2022 (KR) 10-2022-0113906

(51) Int. Cl. C07K 7/06

(52) U.S. Cl. CPC C07K 7/06; C07K 2319/09

(57) ABSTRACT

The present disclosure pertains to: a novel polypeptide composition for intracellular transfection; and uses thereof. This novel polypeptide composition for intracellular transfection has the advantage of greatly improving the transfection efficacy of target substances and having remarkably low cytotoxicity. Accordingly, the polypeptide composition has a variety of uses since various substances can be transported into cells thereby.

### TECHNICAL FIELD

The present disclosure relates to a novel polypeptide for intracellular transfection and uses thereof.

### BACKGROUND ART

Nucleic acid delivery vehicle used for intracellular nucleic acid delivery may be broadly categorized into viral vectors and non-viral vectors.

The non-viral vectors include a variety of formulations such as liposomes, cationic polymers, micelles, emulsions, and nanoparticles. In these formulations, cationic lipids are critical materials in the design of nucleic acid delivery vehicle, as they provide the electrostatic binding force to anionic nucleic acid substances (lipofection). Cationic lipids form complex particles through stable ionic bonds with anionic nucleic acid substances, and the complexes formed in this way are transported into cells through cell membrane fusion or endocytosis.

Conventional cationic lipids have been developed by conjugating neutral fatty acid chains with amine-bearing compounds, such as primary amines, secondary amines, tertiary amines, or quaternary ammonium salts, to impart cationic properties.

It has been reported that the above lipids have relatively high gene transfer effects but are cytotoxic. To mitigate this cytotoxicity, lipids containing amino acid linkers, instead of non-amino acid linkers, have been synthesized.

Recent reports have shown that several cationic lipids, formed by combining fatty acid amines and carboxyl groups of amino acids, exhibit cytotoxicity, contrary to expectations, and in particular, most of the produced cationic lipids have significantly low delivery efficiency for target substances such as oligo-nucleic acids, into cells, thereby showing limited practical applicability. This suggests that it is difficult to obtain intracellular delivery efficiency by simply combining amino acids and fatty acid amines to form a lipid delivery vehicle. Instead, delivery efficiency is determined by the specific structure of the carrier. Therefore, thorough careful preliminary design and experimental results are required prior to its application as a practical delivery system.

In addition, viral vectors are highly efficient for gene delivery, but they are from pathogenic viruses, raising safety concerns and limitations on the size of genes that can be inserted into the vector. Further, the use of viral vectors for nucleic acid delivery is increasingly restricted due to the recent emergence of various issues related to their immunogenicity.

In response to these challenges, the present inventors have developed the present disclosure by creating a novel nucleic acid delivery system.

### DISCLOSURE

### Technical Problem

An object of the present disclosure is to provide a novel polypeptide for intracellular transfection.

Another object of the present disclosure is to provide novel uses of the polypeptide for intracellular transfection.

### Technical Solution

The present disclosure will be described in detail as follows. Meanwhile, each description and embodiment disclosed in the present disclosure may be applied to each of the other descriptions and embodiments. In other words, all combinations of various elements disclosed in the present disclosure fall within the scope of the present disclosure. In addition, it cannot be considered that the scope of the present disclosure is limited by specific descriptions described below.

Further, terms used herein are merely used for illustration purposes, which should not be construed as limiting the present disclosure. Singular expressions include plural expressions unless the context clearly indicates otherwise. In the present specification, terms such as “comprise” or “have” are intended to designate the presence of features, numbers, steps, operations, components, parts, or combinations thereof described in the specification and it should not be understood as precluding the possibility of the presence or addition of one or more other features, numbers, steps, operations, components, parts, or combinations thereof.

Further, unless defined otherwise, all terms used herein, including technical or scientific terms, have the same meaning as commonly understood by one of ordinary skill in the art to which the embodiments belong. Terms such as those defined in commonly used dictionaries should be interpreted as having a meaning consistent with the meaning in the context of the related art, and unless explicitly defined in the present application, it should not to be construed in an idealized or overly formal sense.

In the following specification, description of overlapping content will be omitted to prevent any potential confusion arising from redundancy. In other words, the content of the invention is not limited to the following content; rather, it should be construed in accordance with the comprehensive content of the invention.

### Polypeptide

In one general aspect, there is provided a polypeptide comprising: 9, 10, or 11 consecutive leucines (Leu); and 1, 2, 3, or 4 repeats of peptide of SEQ ID NO: 1 linked thereto.

More specifically, the present disclosure provides a polypeptide essentially consisting of: 9, 10, or 11 consecutive leucines (Leu); and 1, 2, 3, or 4 repeats of peptide of SEQ ID NO: 1 linked thereto.

More specifically, the present disclosure provides a polypeptide consisting of: 9, 10, or 11 consecutive leucines (Leu); and 1, 2, 3, or 4 repeats of peptide of SEQ ID NO: 1 linked thereto.

As used herein, the term “peptide” refers to a molecule formed of amino acid residues linked together by amide bonds (or peptide bonds). The peptide may be synthesized using a gene recombination and protein expression system, preferably in vitro using a peptide synthesizer or the like.

As used herein, the term “polypeptide” refers to a molecule formed of monomers (amino acids) linearly linked by amide bonds (or peptide bonds). The polypeptides include peptides, dipeptides, tripeptides, oligopeptides, etc., which are used to refer to chains formed of two or more amino acids.

As used herein, the terms “amino acid” and “amino acid residue” refer to natural amino acids, non-natural amino acids, and modified amino acids. Unless otherwise noted, all references to amino acids, either generally or specifically by name, include cases to both the D and L stereoisomers (where the structure permits these stereoisomeric forms). Natural amino acids include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val). Non-natural amino acids include modified amino acid residues that are chemically modified at the N-terminal amino group or at the side chain functional group, or that are chemically blocked, either reversibly or irreversibly, such as N-methylated D and L amino acids or residues in which the side chain functional group is chemically modified with another functional group.

The polypeptide according to the present disclosure is a single polypeptide chain comprising fused components, wherein the fused components may be directly or indirectly linked.

The polypeptide according to the present disclosure may include fragments in which an amino acid fragment or part of the peptide is substituted or deleted, a part of the amino acid sequence is modified into a structure capable of increasing stability in vivo, a part of the amino acid sequence is modified to increase hydrophilicity, some or all amino acids are substituted with L- or D-amino acids, or a part of the amino acids is modified, meaning that it includes derivatives thereof as needed.

Specifically, the peptides according to the present disclosure may be in a form in which the N-terminal and/or C-terminal amino acids of the polypeptide are modified to increase the stability and bioactivity of the peptide. For example, the peptides according to the present disclosure may be modified by N-terminal acetylation or C-terminal amidation.

The phrase “linked thereto” according to the present disclosure refers to the linkage and/or binding of a peptide sequence to an N-terminus or C-terminus. Here, the linkage includes both direct bonding or connection via a linker or spacer peptide. Preferably, “linked thereto” may refer to a polypeptide comprising 1, 2, 3 or 4 repeats of peptide of SEQ ID NO. 1 linked to the C-terminus of 9 to 11 consecutive leucines.

In other words, there is provided a polypeptide comprising: 9, 10, or 11 consecutive leucines (Leu); and 1, 2, 3, or 4 repeats of peptide of SEQ ID NO: 1 linked to the C-terminus thereof.

In the present disclosure, SEQ ID NO: 1 is known as a nuclear localization sequence (NLS), comprising the amino acids of PKKKRKV. These peptides are not intended to be simple NLS peptides, but rather are linked to 9, 10, or 11 consecutive leucines to achieve the desirable effect of delivering a target substance into a cell in a combined or integrated manner.

As needed, the peptides may be directly connected or may be connected via a linker or spacer peptide.

The term “linker” or “spacer” refers to a short amino acid sequence used to separate two peptides with different functions in the construction of a polypeptide. The absence of a linker between two or more individual domains within a protein may result in reduced or inappropriate function of the protein domains due to steric hindrance, for example, reduced catalytic activity or binding affinity for receptors/ligands. Artificial linkers may be used to connect protein domains in chimeric proteins, thereby increasing the space between domains. Preferably, the linker or spacer peptide is not particularly limited as long as it exhibits an effect of enhancing the activity of a conjugate of leucine and the peptide of SEQ ID NO: 1 or between repeating peptide bonds of SEQ ID NO: 1. The constituent amino acids may not have any specific biological activity other than to bind the regions together or to preserve some minimum distance or other spatial relationship between these regions, but may be selected to affect some properties of the molecule, such as folding, net charge, and hydrophobicity.

More specifically, the polypeptide may be any one selected from the group consisting of SEQ ID NOs: 2 to 13.

Any of these sequences is shown in Table 1 below.

TABLE 1

| | L₉ | L₁₀ | L₁₁ |
|---|---|---|---|
| 1xNLS | N′- | N′- | N′- |
| | LLLLLLLLLPKKKRKV-C′ | LLLLLLLLLLPKKKRKV- | LLLLLLLLLLLPKKKRKV- |
| | (SEQ ID NO: 2) | C′ (SEQ ID NO: 6) | C′ (SEQ ID NO: 10) |
| 2xNLS | N′- | N′- | N′- |
| | LLLLLLLLLPKKKRKVPKK | LLLLLLLLLLPKKKRKVPK | LLLLLLLLLLLPKKKRKVP |
| | KRKV-C′ (SEQ ID NO: | KKRKV-C′ (SEQ ID | KKKRKV-C′ (SEQ ID |
| | 3) | NO: 7) | NO: 11) |
| 3xNLS | N′- | N′- | N′- |
| | LLLLLLLLLPKKKRKVPKK | LLLLLLLLLLPKKKRKVPK | LLLLLLLLLLLPKKKRKVP |
| | KRKVPKKKRKV-C′ (SEQ | KKRKVPKKKRKV-C′ | KKKRKVPKKKRKV-C′ |
| | ID NO: 4) | (SEQ ID NO: 8) | (SEQ ID NO: 12) |
| 4xNLS | N′- | N′- | N′- |
| | LLLLLLLLLPKKKRKVPKK | LLLLLLLLLLPKKKRKVPK | LLLLLLLLLLLPKKKRKVP |
| | KRKVPKKKRKVPKKKRKV- | KKRKVPKKKRKVPKKKRKV | KKKRKVPKKKRKVPKKKRK |
| | C′ (SEQ ID NO: 5) | -C′ (SEQ ID NO: 9) | V-C′ (SEQ ID NO: |
| | | | 13) |

If necessary, the polypeptide sequence according to the present disclosure may comprise the above-listed SEQ ID NOs: 2 to 13. At this time, a peptide having at least 90% or more, most preferably 95%, 96%, 97%, 98%, 99% or more sequence homology with any one selected from the group consisting of the above SEQ ID NOs: 2 to 13 is also included in the scope of the present disclosure.

The following 9, 10, and 11 consecutive leucines are shown in SEQ ID NOs: 14, 15, and 16, respectively.
9 consecutive leucines: LLLLLLLLL (SEQ ID NO: 14)
10 consecutive leucines: LLLLLLLLLL (SEQ ID NO: 15)
11 consecutive leucines: LLLLLLLLLLL (SEQ ID NO: 16)

In terms of the sequence homology, the present disclosure may comprise a polypeptide having one or more different amino acid residues based on any one selected from the group consisting of SEQ ID NOs: 2 to 13. Amino acid exchanges in proteins and polypeptides that do not alter the overall activity of the molecule are known in the art. The most common exchanges are between amino acid residues Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, or Asp/Gly. In addition, the present disclosure may comprise a peptide having increased structural stability to heat, pH, etc., by mutations or modifications in the amino acid sequence.

The polypeptide according to the present disclosure is a polypeptide for use as a delivery vehicle having the ability to deliver a target substance into a cell.

The polypeptide according to the present disclosure is fused through direct interaction between components and forms a common internal space in the fused particles. The target substance is loaded into this common internal space to deliver the target substance into the cell. In other words, the above-described polypeptide may form a “membrane” to provide an outer layer with an inner compartment to load the target substance. Specifically, the membrane may be formed to create an outer layer, thereby providing an inner compartment to load the target substance.

In particular, its physicochemical properties, variable fusion inducibility, high encapsulation efficiency, high safety and low immunogenicity may be elicited, offering significant potential for application not only in cells but also within the human body.

In addition, the polypeptide having the ability to deliver the target substance according to the present disclosure is a very small peptide, thus minimizing any possible biological interference with the active substance.

Accordingly, in the present disclosure, the polypeptide may be used to deliver the target substance into cells.

As used herein, the term “target substance” refers to any substance capable of exhibiting an activity that modulates intracellular activity by being loaded into a polypeptide and delivered into a cell. Examples of the target substance include, but are not limited to, compounds, proteins, nucleic acids, and the like.

More specifically, the compound may be a low molecular weight compound, a charged high molecular weight compound, or a fluorescent compound.

More specifically, the protein may be at least one selected from the group consisting of an antibody, a receptor-bindable ligand peptide, a protein drug, a cytotoxic polypeptide, a cytotoxic protein, and a fluorescent protein.

More specifically, the nucleic acid may be selected from the group consisting of, for example, DNA, recombinant DNA, plasmid DNA, antisense oligonucleotide, aptamer, RNA, siRNA, shRNA, and miRNA.

The polypeptide according to the present disclosure may be produced using available techniques known in the art. The polypeptide may be synthesized using any suitable procedure known to a person skilled in the art, e.g., any known method for synthesizing polypeptides (e.g., genetic engineering methods, chemical synthesis).

For example, the polypeptide according to the present disclosure may be produced by recombinant techniques according to genetic engineering methods. The production of a peptide by genetic engineering methods comprises, for example, first constructing a nucleic acid (polynucleotide) encoding the polypeptide of the present disclosure or a functional equivalent thereof, using conventional methods. The nucleic acid may be prepared by amplification by PCR using appropriate primers. Alternatively, the DNA sequence may be synthesized by standard methods known in the art, for example, using an automated DNA synthesizer. The constructed nucleic acid may be inserted into a vector comprising one or more expression control sequences (e.g., promoters, enhancers, etc.) operatively linked thereto to regulate expression of the nucleic acid, thereby creating a recombinant expression vector, followed by transfection into host cells. Then, the cells were cultured under media and conditions appropriate for expressing the desired polypeptide, and a substantially pure polypeptide expressed from the nucleic acid was recovered from the culture. The recovery may be performed using methods known in the art. The method is not limited thereto, but may include separation and purification performed by methods known in the art, such as extraction, recrystallization, various types of chromatography (gel filtration, ion exchange, precipitation, adsorption, reversed-phase), electrophoresis, and countercurrent distribution, and the like.

As used above, the term “substantially pure polypeptide” means that the polypeptide according to the present disclosure substantially does not contain any other proteins derived from host cells.

As used herein, the term “vector” refers to a nucleic acid molecule capable of transporting another nucleic acid to a specific target site to which it is associated. Further, the term “expression vector” encompasses plasmids, cosmids, or phages that are capable of facilitating the synthesis of a fusion protein, which is encoded by each recombinant gene carried by the vector.

Furthermore, for example, the polypeptide according to the present disclosure may be produced by chemical synthesis methods known in the art. Representative methods include, but are not limited to, liquid or solid phase synthesis, fragment condensation, F-MOC or T-BOC chemistry.

For example, the polypeptide of the present disclosure may be produced by direct peptide synthesis using solid-phase peptide synthesis (SPPS) method. The solid-phase peptide synthesis (SPPS) method may initiate synthesis by attaching functional units, called linkers, to small porous beads to connect the peptide chain. Unlike liquid-phase methods, peptides form covalent bonds with the beads, preventing loss by the filtration process until the peptides are cleaved from the beads by a specific reactant, such as trifluoroacetic acid (TFA). The synthesis is performed as a cycle (deprotection-wash-coupling-wash), including the protection step that involves the binding of the N-terminal amine of the peptide attached to the solid phase with the N-protected amino acid unit, followed by the deprotection step, and the coupling step, where a new amino acid binds to the exposed amine group. The SPPS method may be performed using microwave technology, which may shorten the time required for coupling and deprotection of each cycle by applying heat during the peptide synthesis process. The heat energy may prevent folding or aggregation of the extended peptide chain and promote chemical bonding.

The present disclosure provides a polynucleotide encoding the polypeptide.

### Use for Delivering Target Substance

In another general aspect, there is provided a composition comprising: a polypeptide comprising 9, 10, or 11 consecutive leucines (Leu), and 1, 2, 3, or 4 repeats of peptide of SEQ ID NO: 1 linked thereto; and a target substance.

In another general aspect, there is provided a composition for intracellular transfection, comprising: a polypeptide comprising 9, 10, or 11 consecutive leucines (Leu), and 1, 2, 3, or 4 repeats of peptide of SEQ ID NO: 1 linked thereto; and a target substance.

In still another general aspect, there is provided a composition for delivering a target substance into the nucleus, comprising: a polypeptide comprising 9, 10, or 11 consecutive leucines (Leu), and 1, 2, 3, or 4 repeats of peptide of SEQ ID NO: 1 linked thereto; and a target substance.

Hereinafter, each of the uses thereof is described in detail.

The present disclosure provides a composition comprising: a polypeptide comprising 9, 10, or 11 consecutive leucines (Leu), and 1, 2, 3, or 4 repeats of peptide of SEQ ID NO: 1 linked thereto; and a target substance. The composition may be used for intracellular transfection purposes.

The composition according to the present disclosure may exhibit various functional effects by transfecting the target substance into a cell and controlling the expression of the target substance in the cell. The term ‘injection’ included in this term is used interchangeably with the expression ‘transportation’, ‘penetration’, ‘transfer’, ‘delivery’, ‘transmission’ or ‘passing through’.

The term “transfection” as used herein means a process of injecting a nucleic acid molecule or protein into a cell, preferably a eukaryotic cell. The nucleic acid molecule may be a genetic sequence encoding a complete protein or a functional portion thereof. The eukaryotic cell may be an animal cell, a mammalian cell, or a human cell, and may be, for example, a stem cell (e.g., an embryonic stem cell, a pluripotent stem cell, an induced pluripotent stem cell, a neural stem cell, a mesenchymal stem cell, a hematopoietic stem cell, a peripheral blood stem cell), a primary cell (e.g., a myoblast, a fibroblast), an immune cell (e.g., a NK cell, a T cell, a dendritic cell, an antigen presenting cell), a cancer cell, an epithelial cell, a skin cell, a gastrointestinal cell, a mucosal cell, or a lung cell.

The composition according to the present disclosure may facilitate the delivery of the target substance into a target cell by increasing the efficiency of transfection into the target cell. The target substance may be delivered into the nucleus as needed.

Therapeutic, imaging, and diagnostic applications are of interest to the composition according to the present disclosure. Thus, by using the polypeptide according to the present disclosure, various target substances may be easily encapsulated internally in a test tube, in a living body, and preferably in a human body, allowing for the customized transfection of target substances into cells.

The polypeptide according to the present disclosure may interact with each other to form a spherical shape and load the target substance inside. The spherical shape has a diameter in the range of about 30 nm to 200 nm, more preferably in the range of 50 nm to 150 nm, and even more preferably in the range of 60 to 130 nm. The polypeptide may contain the target substance inside.

In particular, as a result of measuring the hydrodynamic radius and zeta potential of the produced polypeptide carrier using a particle size analyzer, the hydrodynamic radius was confirmed to be 30 nm to 50 nm, and the zeta potential was confirmed to be about +2 mV to +6 mV. This demonstrates that a nano-sized complex is capable of being formed using the polypeptide and target substance according to the present disclosure, with the desired size being consistently maintained, regardless of whether the target substance is loaded. In addition, it was confirmed that the target substance is loaded at a desired level or higher without affecting hybridization, thereby exhibiting delivery efficacy.

For example, the polypeptide according to the present disclosure may introduce a target substance into a cell through a general method known in the art. Such an introduction method may be performed within the level of generally known cell culture conditions.

For example, mixing with a target substance in vitro may be performed to induce transfection into a cell. The mixing may be performed under conditions of 32 to 40° C., preferably about 37° C., for 10, 20, 30, 40, 50 or 60 minutes, 2 hours, 3 hours, 4 hours, 5 hours, 6 hours or more. In addition, a buffer for the mixing may include any known medium or the like. Examples of media capable of being used may include, but are not limited to, Opti-MEM, DPBS, and/or RPMI-1640.

In addition, the polypeptide according to the present disclosure may be mixed with the target substance under ex vivo and in vivo conditions to allow the target substance to reach the target cell. In the present disclosure, the transfection of the target substance may be performed for 0.5, 1, 2, 3, 4, 5 or more hours.

In some embodiments, the composition (polypeptide and target substance) may be pre-incubated with the polypeptide and target substance to form a mixture prior to contacting the target cell.

The method may also comprise multiple treatments of the composition to the cells (e.g., 1, 2, 3, 4 or more times per day, and/or on a predetermined schedule). In this case, a lower concentration of the composition may be recommended (e.g., for reduced toxicity). In some embodiments, the cell may be a suspension cell or an adherent cell. In some embodiments, a person skilled in the art may adapt the teachings of the present description using different combinations of transporters, domains, uses, and methods to suit the specific needs of delivering the target substance to specific cells with desired viability.

For example, the target substance may be a compound, a protein, a nucleic acid, etc.

More specifically, the compound may be a low molecular weight compound, a charged high molecular weight compound, or a fluorescent compound.

More specifically, the protein may be at least one selected from the group consisting of an antibody, a receptor-bindable ligand peptide, a protein drug, a cytotoxic polypeptide, a cytotoxic protein, and a fluorescent protein.

In detail, the nucleic acid may be selected from the group consisting of, for example, DNA, recombinant DNA, plasmid DNA, antisense oligonucleotide, aptamer, RNA, siRNA, shRNA, and miRNA.

The target substance may be delivered into cells or the human body while changing the target substance depending on the purpose of treatment or prevention, experimental purpose for research and development, etc. Examples of possible target substances may include any substance that can be considered for examination of various activities, such as endogenous ligands, neurotransmitters, hormones, autacoids, cytokines, antiviral agents, anticancer agents, antibiotics, oxygen-enhancing agents, oxygen-containing agents, antiepileptic agents, and anti-inflammatory drugs.

Transfection of a target substance using a composition according to the present disclosure may significantly increase the delivery efficiency into cells compared to general transfection. This enables the composition according to the present disclosure to deliver the target substance into cells, thereby exhibiting therapeutic efficacy in treating a disease or disorder. According to an embodiment of the present disclosure, the polypeptide according to the present disclosure may deliver large amounts of the target substance into cells.

The present disclosure provides a method for delivering a target substance into a cell, comprising contacting the cell with the composition described above.

The cell may be an animal cell, a mammalian cell, or a human cell, and may be, for example, but is not limited to, a stem cell (e.g., an embryonic stem cell, a pluripotent stem cell, an induced pluripotent stem cell, a neural stem cell, a mesenchymal stem cell, a hematopoietic stem cell, a peripheral blood stem cell), a primary cell (e.g., a myoblast, a fibroblast), an immune cell (e.g., a NK cell, a T cell, a dendritic cell, an antigen presenting cell), a cancer cell, an epithelial cell, a skin cell, a gastrointestinal cell, a mucosal cell, or a lung cell.

### Therapeutic Uses

In another general aspect, there is provided a composition for drug delivery, comprising: a polypeptide comprising 9, 10, or 11 consecutive leucines (Leu), and 1, 2, 3, or 4 repeats of peptide of SEQ ID NO: 1 linked thereto; and a target substance.

In another general aspect, there is provided a drug adjuvant composition, comprising: a polypeptide comprising 9, 10, or 11 consecutive leucines (Leu), and 1, 2, 3, or 4 repeats of peptide of SEQ ID NO: 1 linked thereto; and a target substance.

In another general aspect, there is provided a composition for preventing or treating a disease, comprising: a polypeptide comprising: 9, 10, or 11 consecutive leucines (Leu) and 1, 2, 3, or 4 repeats of peptide of SEQ ID NO: 1 linked thereto; and a drug.

In another general aspect, there is provided a composition for preventing or treating cancer, comprising: a polypeptide comprising 9, 10, or 11 consecutive leucines (Leu), and 1, 2, 3, or 4 repeats of peptide of SEQ ID NO: 1 linked thereto; and a target substance.

As used herein, the term “drug delivery use (or referred to as for drug delivery)” refers to the use as a delivery substance designed to transport a drug into a target cell.

The term “drug adjuvant use (or referred to as for drug adjuvant)” refers to the use as a pharmaceutical aid used in combination with a drug to maximize the effect of a conventional drug.

The drug delivery or drug adjuvant use means a use that has a relatively low medicinal effect when administered alone, but significantly improves the efficacy of the drug when administered together with the polypeptide according to the present disclosure.

In the present disclosure, the composition may be used to deliver a target substance into a living tissue or blood. The composition may be delivered through cells or intercellular junctions constituting living tissues, but there is no limitation on the delivery method.

The living tissue refers to one or more epithelial tissues, muscle tissues, nerve tissues, and connective tissues, and each organ may be composed of one or more tissues, and thus, may include various living organs such as, but not limited to, mucosa, skin, brain, lung, liver, kidney, spleen, pulmonary organ, heart, stomach, large intestine, digestive tract, bladder, ureter, urethra, ovaries, testes, genitals, muscles, blood, blood vessels, lymph vessels, lymph nodes, thymus, pancreas, adrenal glands, thyroid, parathyroid glands, larynx, tonsils, bronchial tubes, and alveoli.

In particular, the compositions of the present disclosure may deliver a conventionally targeted substance, such as a biologically active substance, into a cell for direct action, and may also target any one or more immune cells selected from the group consisting of macrophages, B lymphocytes, T lymphocytes, mast cells, monocytes, dendritic cells, eosinophils, natural killer cells, basophils, and neutrophils among the cells to deliver the biologically active substance to act within the immune cell. Furthermore, in contrast to conventional techniques, which relied on viral vectors to deliver genes to immune cells, the composition according to the present disclosure may also deliver genes to immune cells via non-viral vectors, which may represent a breakthrough in the development of drug delivery systems.

In the present disclosure, the target substance refers to any substance that is intended to be delivered into a cell, as described above, as a functional modulating substance having biological activity to regulate any physiological phenomenon in vivo by being loaded in a polypeptide and delivered into the cell.

The drug may be selected from the group consisting of, but is not limited to, a compound drug, a bio drug, a nucleic acid drug, a peptide drug, a protein drug, a hormone, a contrast agent, and an antibody. Preferably, the nucleic acid may be selected from the group consisting of DNA, recombinant DNA, plasmid DNA, antisense oligonucleotide, aptamer, RNA, siRNA, shRNA, and miRNA.

The drug according to the present disclosure may be a nucleic acid drug or an antibody.

Specifically, the drug may be a substance that does not easily move into cells through general routes, or that has low specific delivery efficiency even if it moves into cells easily. More specifically, the drug may be an antibody that is not easily delivered in most cells, or a genetic material such as a plasmid, mRNA, or siRNA that is difficult to deliver in immune cells, stem cells, or neural cells.

The present disclosure may be applied to a method for intracellularly delivering a target substance (preferably a drug) to cells in a living body. This may be accomplished by parenteral administration or direct injection into a tissue, organ, or system.

In other words, the composition according to the present disclosure may be used in mammals, preferably humans, and may deliver the target substance intracellularly, for example by administration via intravenous, intraperitoneal, intramuscular, subcutaneous, intradermal, nasal, mucosal, inhalation, and oral routes.

As used herein, the term “treatment” means suppression or alleviation of a disease or condition. Thus, the term “therapeutically effective amount” as used herein means an amount sufficient to achieve the pharmacologic effect.

The composition may be formulated and provided in an appropriate form. The preparation of the present disclosure may be formulated and used in oral formulations such as powders, granules, tablets, capsules, ointments, suspensions, emulsions, syrups, and aerosols, or parenteral formulations such as transdermal preparations, suppositories, and sterile injectable solutions, respectively, according to conventional methods.

Further, the preparation may further comprise pharmaceutically suitable and physiologically acceptable auxiliaries such as carriers, excipients and diluents. Examples of the carrier, excipient and diluent that may be contained in the pharmaceutical composition of the present disclosure may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil. When formulating the composition, commonly used diluents or excipients such as fillers, extenders, binders, wetting agents, disintegrants, surfactants, and the like may be used.

More specifically, the preparation may comprise a carrier for adding to and formulating the pharmaceutical composition (active ingredient). The carrier may include binders, glidants, suspending agents, solubilizers, buffers, preservatives, lubricants, isotonic agents, excipients, stabilizers, dispersants, suspending agents, colorants, flavorings, etc.

The composition may be administered alone, but in general, may be administered mixed with a pharmaceutical carrier selected in consideration of the administration manner and standard pharmaceutical practice.

When provided for parenteral use, the dosage form may be a liquid dosage form, such as a liquid, gel, cleansing composition, tablet for insertion, suppository form, topical administration such as cream, ointment, dressing solution, spray, and other coating agents, a solution form, a suspension form, an emulsion type, etc., and may comprise an external skin preparation such as a sterilized aqueous solution, non-aqueous solvent, suspension, emulsion, freeze-dried formulation, suppository, cream, ointment, jelly, foam, detergent or insert, preferably a liquid or gel, cleansing composition, and a tablet for insertion, etc. The formulation may be prepared, for example, by adding a solubilizing agent, emulsifier, buffer for pH adjustment, etc., to sterile water. As the non-aqueous solvent and the suspension, propylene glycol, polyethylene glycol, vegetable oils such as olive oil, and injectable esters such as ethyl oleate, etc., may be used.

Furthermore, if the preparation is provided for oral administration, it may be administered orally, intra-orally, or under the tongue, for example, in the form of a tablet containing starch or lactose, or in the form of a capsule alone or containing an excipient, or in the form of an elixir or suspension containing a chemical agent for flavoring or coloring.

The dosage of the preparation may vary depending on the patient's age, weight, sex, dosage form, health status, and disease severity, and may be administered once a day or several times a day at regular intervals as determined by the physician or pharmacist. For example, the daily dosage may be, based on the active ingredient content, 0.001 to 10000 mg/kg, 0.01 to 10000 mg/kg, 0.1 to 10000 mg/kg, 0.5 to 10000 mg/kg, 0.001 to 1000 mg/kg, 0.01 to 1000 mg/kg, 0.1 to 1000 mg/kg, 0.5 to 1000 mg/kg, 0.001 to 500 mg/kg, 0.01 to 500 mg/kg, 0.1 to 500 mg/kg, 0.5 to 500 mg/kg, 0.001 to 300 mg/kg, 0.01 to 300 mg/kg, 0.1 to 300 mg/kg, or 0.5 to 300 mg/kg. The above dosage is an example of an average case, and may be higher or lower depending on individual differences.

It is recommended that the daily dosage of the preparation be in the above range, as a dosage below this range is unlikely to produce a significant effect, and exceeding this dosage is not only uneconomical but also outside the range of commercially available dosages and may cause undesirable side effects.

The subject to which the composition is administered may be a mammal, such as a human, a cell, tissue, and fluid isolated from the mammal, or a culture thereof.

Further, the present disclosure provides a composition for use in producing a genetically modified cell, the composition comprising: a polypeptide comprising 9, 10, or 11 consecutive leucines (Leu) and 1, 2, 3, or 4 repeats of peptide of SEQ ID NO: 1 linked thereto; and a nucleic acid molecule encoding a chimeric antigen receptor (CAR), or a nucleic acid construct comprising the nucleic acid molecule.

More specifically, the chimeric antigen receptor (CAR) described herein may be produced by any means known in the art, even though preferably they are produced using recombinant DNA technology. Nucleic acids encoding several regions of the chimeric receptor may be prepared, and conveniently, may be assembled into a complete encoding sequence by standard techniques of molecular cloning known in the art (e.g., genomic library screening, PCR, primer-assisted ligation, site-directed mutagenesis, etc.). The obtained encoding region is preferably inserted into an expression vector and used to transform a suitable expression host cell line, an immune cell line, preferably a T lymphocyte cell line, and most preferably an autologous T lymphocyte cell line.

The nucleic acid construct comprises an expression vector comprising a nucleic acid sequence encoding the chimeric antigen receptor described above.

The nucleic acid molecule may comprise any polyribonucleotide or polydeoxyribonucleotide, which may be unmodified or modified, RNA or DNA. For example, the nucleic acid molecule may comprise single- and/or double-stranded DNA, DNA that is a mixture of single- and double-stranded regions, single- and double-stranded RNA, and RNA that is a mixture of single- and double-stranded regions, a hybrid molecule comprising DNA and RNA that may be single-stranded or, more typically, double-stranded or a mixture of single- and double-stranded regions. Further, the nucleic acid molecule may comprise a triple-stranded region comprising RNA or DNA or both RNA and DNA. The nucleic acid molecule may also comprise one or more modified bases or a DNA or RNA backbone modified for stability or other reasons. Since various modifications can be made to DNA and RNA; the term “nucleic acid molecule” includes chemically, enzymatically, or metabolically modified forms.

It should be understood that the nucleic acid product may further comprise one or more of the following: an origin of replication for one or more hosts; a selectable marker gene active in one or more hosts; and/or one or more transcriptional control sequences, wherein expression of the nucleic acid molecule is under the control of the transcriptional control sequences. The term “selectable marker gene” as used herein refers to any gene that facilitates identification and/or selection of cells that are transfected or injected with the construct by conferring a phenotype upon expression in the cells. The “selectable marker gene” includes any nucleotide sequence that, when expressed by a cell transfected with the construct, confers a phenotype on the cell that facilitates identification and/or selection of such transfected cells. A wide variety of nucleotide sequences encoding suitable selectable markers are known in the art. Exemplary nucleotide sequences encoding selectable markers include: among other sequences that allow for optimal selection of cells using techniques such as fluorescence-activated cell sorting (FACS), adenosine deaminase (ADA) gene; cytosine deaminase (CDA) gene; dihydrofolate reductase (DHFR) gene; histidinol dehydrogenase (hisD) gene; puromycin-N-acetyl transferase (PAC) gene; thymidine kinase (TK) gene; xanthine-guanine phosphoribosyltransferase (XGPRT) gene or antibiotic resistance genes such as ampicillin-resistance gene, puromycin-resistance gene, bleomycin-resistance gene, hydromycin-resistance gene, kanamycin-resistance gene, and ampicillin-resistance gene; fluorescent reporter genes such as green, red, yellow or blue fluorescent protein-encoding genes; and luminescence-based reporter genes such as luciferase gene.

As described above, the nucleic acid construct may also comprise one or more transcriptional control sequences. As used herein, the term “transcriptional control sequence” is to be understood to include any nucleic acid sequence that affects the transcription of an operably linked nucleic acid. The transcriptional control sequence may include, for example, a leader, a polyadenylation sequence, a promoter, an enhancer or upstream activating sequence, and a transcription terminator.

Typically, the transcriptional control sequence comprises at least a promoter. As used herein, the term “promoter” describes any nucleic acid that confers, activates, or enhances the expression of a nucleic acid in a cell. The transcriptional control sequence is considered “operably linked” to a given nucleic acid molecule if the transcriptional control sequence is capable of promoting, repressing, or otherwise modulating transcription of the nucleic acid molecule.

The nucleic acid molecule is under the control of a transcriptional control sequence, for example, a constitutive promoter or an inducible promoter. A promoter may constitutively regulate the expression of a nucleic acid molecule to which it is operably linked, or may differentially regulate the expression with respect to the cell, tissue, or organ in which expression occurs. Thus, the promoter may comprise, for example, a constitutive promoter, or an inducible promoter. The “constitutive promoter” is a promoter that is active under most environmental and physiological conditions. The “inducible promoter” is a promoter that is active under certain environmental or physiological conditions. The present disclosure contemplates the use of any promoter active within the desired cell. Thus, a wide array of promoters may be readily assumed by a person skilled in the art. A mammalian constitutive promoter may include, but is not limited to, Simian virus 40 (SV40), cytomegalovirus (CMV), P-antin, ubiquitin C (UBC), elongation factor-1 alpha (E3A), phosphoglycerate kinase (PGK), and CMV early enhancer/chicken β-actin (CAGG). The inducible control sequence may also comprise a terminator. As used herein, the term “terminator” refers to a DNA sequence at the end of a transcription unit that signals the termination of transcription. The terminator is a 3′-non-translated DNA sequence that typically contains a polyadenylation signal, which facilitates the addition of a polyadenylate sequence to the 3′-end of the primary transcript. As with the promoter sequence, the terminator may be any terminator sequence that is operable in the cell, tissue or organ where it is intended to be used. Suitable terminators will be known to those skilled in the art.

The nucleic acid construct according to the present disclosure may further comprise additional sequences, for example sequences allowing for enhanced expression, cytoplasmic or membrane transport, and location signals.

The present disclosure extends essentially to all genetic constructs as described herein. Such constructs may further comprise nucleotide sequences intended for maintenance and/or replication of the genetic construct in a eukaryotic cell and/or integration of the genetic construct or a portion thereof into the genome of the eukaryotic cell. The nucleic acid construct may be in any suitable form, such as a plasmid, phage, transposon, cosmid, chromosome, vector, etc., which, when associated with appropriate control elements, is capable of replicating and transferring the genetic sequence contained within the construct between cells.

In at least some embodiments, the present disclosure provides a nucleic acid molecule, or nucleic acid construct, encoding the CAR as described above for use in producing a genetically modified cell.

Further, in at least some embodiments, the present disclosure provides use of a nucleic acid molecule in the preparation of a vector for transformation, transfection, or transduction of a cell. Preferably, the cell is a T cell expressing one or more of CD3, CD4 or CD8. Cells suitable for genetic modification may be xenogeneic or autologous.

The nucleic acid molecule encoding the chimeric antigen receptor described above, or the nucleic acid construct comprising the nucleic acid molecule, may be injected intracellularly via a polypeptide according to the present disclosure. Such intracellular introduction may result in CAR-transfected cells with enhanced therapeutic efficacy as it achieves intracellular transfection with higher efficiency compared to known intracellular transfection methods.

Thus, the present disclosure provides a composition for preventing or treating cancer, comprising: a polypeptide comprising 9, 10, or 11 consecutive leucines (Leu) and 1, 2, 3, or 4 repeats of peptide of SEQ ID NO: 1 linked thereto; and a nucleic acid molecule encoding a chimeric antigen receptor, or a nucleic acid construct comprising the nucleic acid molecule.

More specifically, the present disclosure provides a composition for preventing or treating cancer, the composition comprising: a genetically modified cell with the polypeptide that comprises 9, 10, or 11 consecutive leucines (Leu) and 1, 2, 3, or 4 repeats of peptide of SEQ ID NO: 1 linked thereto, and the nucleic acid molecule encoding a chimeric antigen receptor, or the nucleic acid construct comprising the nucleic acid molecule.

In other words, there is provided the use of the genetically modified cell as described above for preventing or treating cancer. Therefore, the present disclosure provides a method of preventing or treating a patient having cancer, the method comprising: introducing a chimeric antigen receptor-expressing cell into a patient for exposure.

In the present disclosure, the cell may be preferably a T cell, or an NK cell. More preferably, the cell is a T cell expressing one or more of CD3, CD4 or CD8.

The cancer may be at least one selected from the group consisting of bladder cancer, brain cancer, breast cancer, cervical cancer, colon cancer, endometrial cancer, epithelial cancer, oesophageal cancer, lung cancer, mouth cancer, ovarian cancer, kidney cancer, liver cancer, leukemia, lymphoma, myeloma, pancreatic cancer, prostate cancer, rectal cancer, skin cancer, stomach cancer, testicular cancer, thyroid cancer, and tongue cancer.

The therapeutic composition may be administered about once to about five times per week. In some embodiments, the composition is administered once. In some embodiments, the composition is administered twice. In some embodiments, the composition is administered three times. In some embodiments, the composition is administered four times. In some embodiments, the composition comprises at least 5×10⁸ cells.

According to an embodiment of the present disclosure, the polypeptide according to the present disclosure was employed to load the CAR DNA plasmid thereon, resulting in the production of CAR-T cells, and the therapeutic efficacy of these cells against cancer was subsequently validated. These CAR-T cells typically have poor transformation efficiency. However, the use of the polypeptide according to the present disclosure as a delivery system exhibits high delivery efficacy for plasmid DNA, which enables mass production of CAR-T cells with excellent therapeutic efficacy.

The present disclosure provides a method of treating or preventing a disease or disorder, comprising administering a therapeutically effective amount of the composition to a subject in need thereof.

The present disclosure provides a method for delivering a target substance into a cell, comprising treating the cell with the composition described above.

The present disclosure provides a method for delivering a target substance into a cell, comprising treating a subject with the polypeptide comprising the composition described above.

The present disclosure provides a method for selectively delivering a target substance into a cell, comprising treating the cell with the composition described above.

The present disclosure provides a method for selectively delivering a target substance into a cell, comprising treating a subject with the composition described above.

The present disclosure provides a composition comprising a target substance for use in the treatment or prevention of the disease or disorder.

The present disclosure provides a composition comprising a polypeptide for delivering a target substance into a cell.

The present disclosure provides uses of the polypeptide in the manufacture of a preparation for delivering a target substance into a cell.

The present disclosure also provides uses of the above-described compositions and utilization methods thereof.

### Advantageous Effects

This novel polypeptide composition for intracellular transfection has the advantage of greatly improving the transfection efficacy of target substances and having remarkably low cytotoxicity. Accordingly, the polypeptide composition has a variety of uses since various substances can be transported into cells thereby.

### DESCRIPTION OF DRAWINGS

FIG. 1 shows the plasmid DNA delivery efficiency to Jurkat T cells for peptides (Peptides 1 to 10) containing various types of hydrophobic amino acids.

FIG. 2 shows the plasmid DNA delivery efficiency to Jurkat T cells for peptides (Peptides 1 and 11) with different lengths of leucine, i.e., 10 or 11 leucines.

FIG. 3 shows the plasmid DNA delivery efficiency to Jurkat T cells for peptides (Peptides 1, 12, and 13) produced with 2, 3, or 4 different copies of NLS.

FIG. 4 shows the plasmid DNA delivery efficiency to Jurkat T cells for peptides (Peptides 1 and 14) with NLS sequence located at the C-terminus or N-terminus.

FIG. 5 shows the change in expression of GFP depending on L₁₀-2×NLS concentration, confirmed by immunoblotting.

FIG. 6 shows the change in expression of GFP depending on pEGFP-N3 concentration, confirmed by immunoblotting.

FIG. 7 shows the change in expression of GFP depending on the mixing temperature of L₁₀-2×NLS and pEGFP-N3, confirmed by immunoblotting.

FIG. 8 shows the change in expression of GFP depending on the mixing time of L₁₀-2×NLS and pEGFP-N3, confirmed by immunoblotting.

FIG. 9 shows the change in expression of GFP depending on the type of buffer used to mix L₁₀-2×NLS and pEGFP-N3, confirmed by immunoblotting.

FIG. 10 shows the change in expression of GFP depending on an incubation time of transfection, confirmed by immunoblotting.

FIG. 11 shows the size of the L₁₀-2×NLS and plasmid DNA complex.

FIG. 12 shows the zeta potential of the L₁₀-2×NLS and plasmid DNA complex.

FIG. 13 is a transmission electron microscopy (TEM) image showing the morphology of the L₁₀-2×NLS and plasmid DNA complex.

FIG. 14 shows the change in morphology of Jurkat T cells after transfection of plasmid DNA with L₁₀-2×NLS.

FIG. 15 shows the change in viability of Jurkat T cells after transfection of plasmid DNA with L₁₀-2×NLS.

FIG. 16 shows the plasmid DNA delivery efficiency into Jurkat T cells, using L₁₀-2×NLS.

FIG. 17 shows the location of the plasmid DNA, delivered using L₁₀-2×NLS, in Jurkat T cells.

FIG. 18 shows the quantification of the location of plasmid DNA, delivered using L₁₀-2×NLS, in Jurkat T cells.

FIG. 19 shows the gene delivery effect into human primary T cells, using L₁₀-2×NLS.

FIG. 20 shows the activity efficacy of CAR-T produced using L₁₀-2×NLS.

FIG. 21 shows the antibody delivery effect into Jurkat T cells, using L₁₀-2×NLS.

FIG. 22 shows the presence of antibodies in Jurkat T cells, using L₁₀-2×NLS.

### BEST MODE

Hereinafter, preferred Examples are presented to facilitate understanding of the present disclosure. These Examples are only provided to more easily understand the present disclosure, and do not impose limitations on the content of the present disclosure.

### Example 1. Amino Acid Sequences of Peptides Optimized for Plasmid DNA Delivery

### 1-1. Plasmid DNA Delivery Effect Depending on Hydrophobic Amino Acid Type

Each peptide was synthesized by conjugating a sequence comprising two copies of the nuclear localization sequence (NLS, PKKKRKV) (2×NLS) to a sequence linked with leucine (L), phenylalanine (F), methionine (M), valine (V), glycine (G), proline (P), alanine (A), tyrosine (Y), isoleucine (I) and/or tryptophan (W).

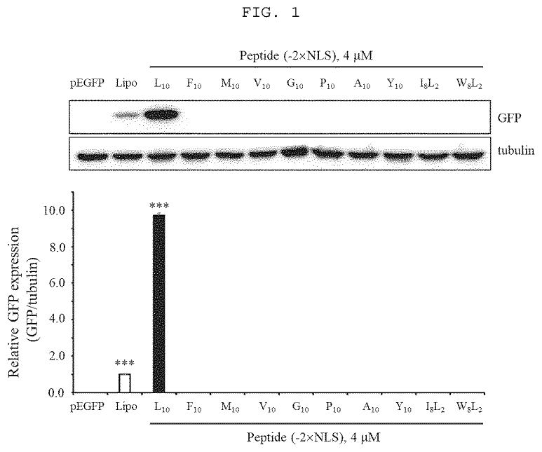

Specific sequences thereof are shown in Table 2 below (Peptides 1 to 10). Each of the above peptides at a concentration of 4 μM was mixed with 2 μg of pEGFP-N3 plasmid for 30 minutes in a CO₂ incubator at 37° C. After transfection of the plasmids into Jurkat T cells for 30 minutes, the expression of GFP was analyzed by immunoblotting after 24 hours (FIG. 1).

As a result, the L₁₀-2×NLS peptide composed of leucine was confirmed to have the highest GFP expression. In particular, the transfection showed a significantly higher level compared to Lipofectamine®, which is generally used for transfection.

### 1-2. Plasmid DNA Delivery Effect Depending on Leucine Amino Acid Length

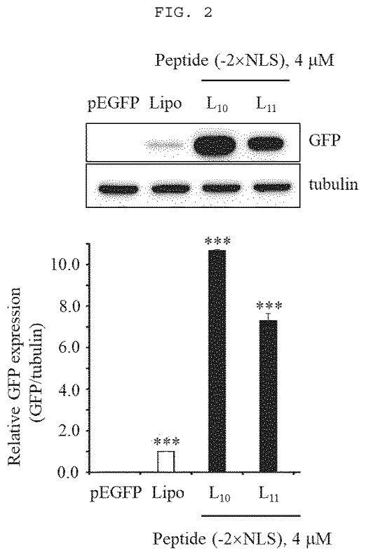

Peptides containing 10 or 11 leucine amino acids and 2×NLS linked thereto were prepared (Peptides 1 and 11), and based on these peptides, the effect of the number of leucine amino acids on the transmission level was further determined. Each of the above peptides was subjected to the same experiment as in Example 1-1, and then the expression of GFP was analyzed by immunoblotting (FIG. 2).

As a result, it was confirmed in view of the length that the highest efficiency was achieved with 10 leucines, and that a certain level of high efficiency was maintained even with 11 leucines.

### 1-3. Plasmid DNA Delivery Effect Depending on Copy Number and N-Terminal/C-Terminal Location of NLS

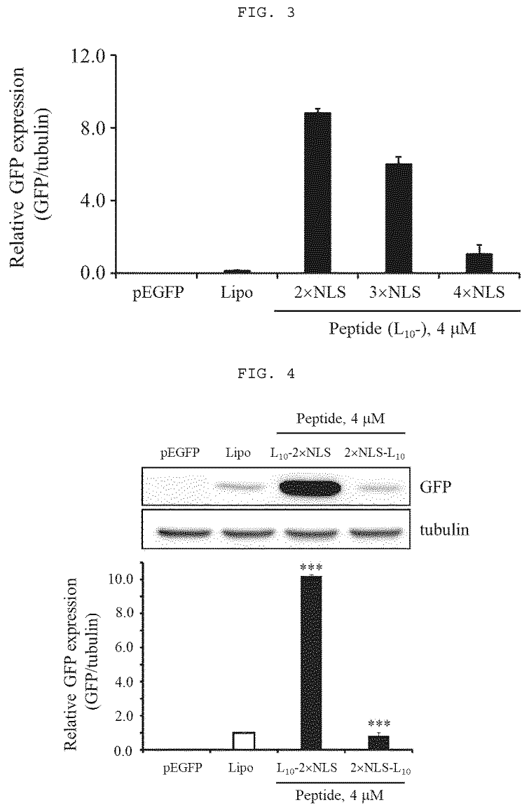

Peptides were prepared by varying the copy number of NLS, located following the 10 leucine amino acid sequences, to 2, 3, or 4, and specific sequences thereof are shown in Table 2 below (Peptides 1, 12, and 13). For each peptide, the change in GFP expression depending on the copy number of NLS was confirmed by immunoblotting (FIG. 3).

As a result, the transfection of pEGFP-N3 into Jurkat T cells using L₁₀-2×NLS showed the highest expression of GFP. In addition, it was confirmed that expression levels were maintained above a certain level even when the number of NLS copies was increased to 3 and 4.

Accordingly, the copy number of NLS was set to 2, and a peptide (L₁₀-2×NLS) in which the NLS amino acid sequence was located at the C-terminus, following leucine, and a peptide (2×NLS-L₁₀) in which the NLS amino acid sequence was located at the N-terminus, preceding leucine, were prepared. Each of the above peptides (Peptide 1 and 14) was mixed with pEGFP-N3 and transfected into Jurkat T cells. Then, the expression changes of GFP were compared. Upon examining the transformation efficiency, it was confirmed that the peptide (L₁₀-2×NLS) with the NLS amino acid sequence located at the C-terminus following leucine, exhibited high efficiency (FIG. 4).

In other words, it was confirmed that a peptide optimized for plasmid DNA delivery into Jurkat T cells contained around 10 leucine amino acid sequences and exhibited above a certain level of transfection efficacy at the level of 2 to 4 copies of NLS. Furthermore, it was shown that L₁₀-2×NLS, located at the C-terminus of leucine, contained the amino acid sequence of the peptide optimized for plasmid DNA delivery to Jurkat T cells.

The specific sequences of Peptides 1 to 14 used in Example 1 are summarized in Table 2 below, and each peptide had modification by N-terminal acetylation (N′ acetylation) or C-terminal amidation (C′ amidation) to increase the stability and bioactivity of the peptide.

TABLE 2

| Peptide | Name | Sequence | Modifications |
|---|---|---|---|
| 1 | L₁₀-2xNLS | N′-LLLLLLLLLLPKKKRKVPKKKRKV-C′ | N′ |
| | | (SEQ ID NO: 7) | acetylation/C′ |
| | | | amidation |
| 2 | F₁₀-2xNLS | N′-FFFFFFFFFFPKKKRKVPKKKRKV-C′ | N′ acetylation/C′ |
| | | (SEQ ID NO: 17) | amidation |
| 3 | M₁₀-2xNLS | N′-MMMMMMMMMMPKKKRKVPKKKRKV-C′ | N′ |
| | | (SEQ ID NO: 18) | acetylation/C′ |
| | | | amidation |
| 4 | V₁₀-2xNLS | N′-VVVVVVVVVVPKKKRKVPKKKRKV-C′ | N′ |
| | | (SEQ ID NO: 19) | acetylation/C′ |
| | | | amidation |
| 5 | G₁₀-2xNLS | N′-GGGGGGGGGGPKKKRKVPKKKRKV-C′ | N′ |
| | | (SEQ ID NO: 20) | acetylation/C′ |
| | | | amidation |
| 6 | P₁₀-2xNLS | N′-PPPPPPPPPPPKKKRKVPKKKRKV-C′ | N′ |
| | | (SEQ ID NO: 21) | acetylation/C′ |
| | | | amidation |
| 7 | A₁₀-2xNLS | N′-AAAAAAAAAAPKKKRKVPKKKRKV-C′ | N′ |
| | | (SEQ ID NO: 22) | acetylation/C′ |
| | | | amidation |
| 8 | Y₁₀-2xNLS | N′-YYYYYYYYYYPKKKRKVPKKKRKV-C′ | N′ |
| | | (SEQ ID NO: 23) | acetylation/C′ |
| | | | amidation |
| 9 | I₈L₂-2xNLS | N′-IIIILIIIILPKKKRKVPKKKRKV-C′ | N′ |
| | | (SEQ ID NO: 24) | acetylation/C′ |
| | | | amidation |
| 10 | W₈L₂-2xNLS | N′-WWWWLWWWWLPKKKRKVPKKKRKV-C′ | N′ |
| | | (SEQ ID NO: 25) | acetylation/C′ |
| | | | amidation |
| 11 | L₁₁-2xNLS | N′-LLLLLLLLLLLPKKKRKVPKKKRKV-C′ | N′ |
| | | (SEQ ID NO: 11) | acetylation/C′ |
| | | | amidation |
| 12 | L₁₀-3xNLS | N′-LLLLLLLLLLPKKKRKVPKKKRKVPKK | N′ |
| | | KRKV-C′ | acetylation/C′ |
| | | (SEQ ID NO: 8) | amidation |
| 13 | L₁₀-4xNLS | N′-LLLLLLLLLLPKKKRKVPKKKRKVPKK | N′ |
| | | KRKVPKKKRKV-C′ | acetylation/C′ |
| | | (SEQ ID NO: 9) | amidation |
| 14 | 2xNLS-L₁₀ | N′-PKKKRKVPKKKRKVLLLLLLLLLL-C′ | N′ |
| | | (SEQ ID NO: 26) | acetylation/C′ |
| | | | amidation |

### Example 2. Optimized Conditions for Intracellular Transfection of Plasmid DNA

2-1. Plasmid DNA Delivery Effect Depending on L₁₀-2×NLS and pEGFP-N3 Concentration

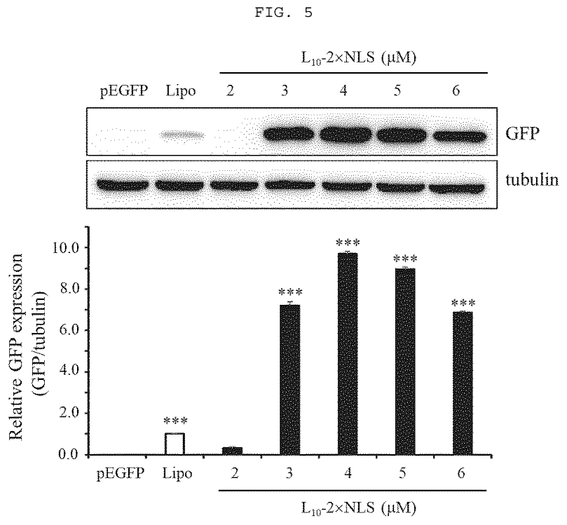

L₁₀-2×NLS at different concentrations (2, 3, 4, 5, or 6 μM) was mixed with 2 μg of pEGFP-N3 and transfected into Jurkat T cells. The expression of GFP was then confirmed by immunoblotting (FIG. 5).

As a result, it was found that L₁₀-2×NLS at a concentration of 4 μM showed the highest expression efficiency of GFP.

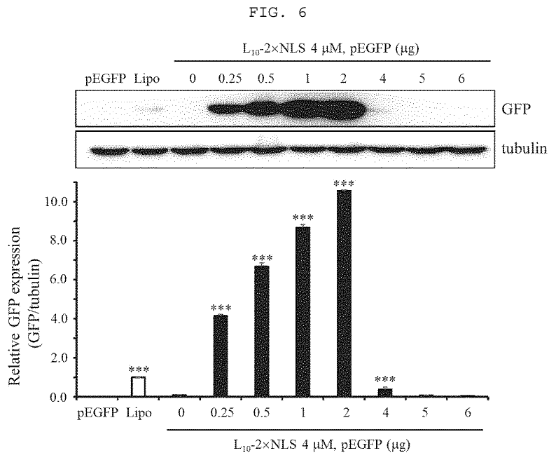

Next, pEGFP-N3 at different concentrations (0, 0.25, 0.5, 1, 2, 4, 5, or 6 μg) was mixed with 4 μM of L₁₀-2×NLS and transfected into Jurkat T cells. The expression of GFP was then confirmed by immunoblotting (FIG. 6).

As a result, the expression efficiency of GFP was highest when pEGFP-N3 was 2 μg.

The following experiments were performed considering the concentration range of injection for the desired transformation.

2-2. Plasmid DNA Delivery Effect Depending on Mixing Conditions of L₁₀-2×NLS and pEGFP-N3

To determine the most effective mixing conditions for plasmid DNA delivery, the temperature, time, and buffer used for mixing 4 μM L₁₀-2×NLS and 2 μg pEGFP-N3 were varied, and the gene transfer effects under each condition were compared.

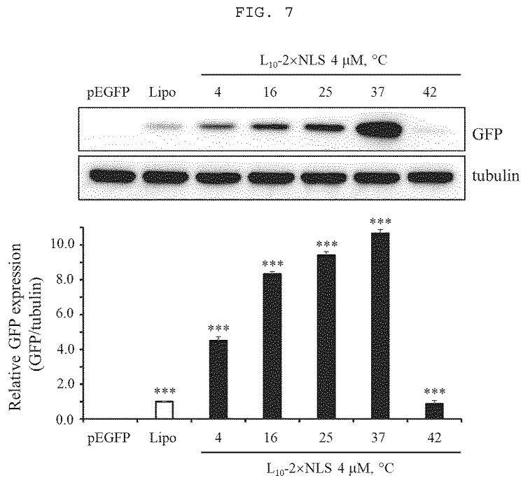

First, 4 μM L₁₀-2×NLS and 2 μg pEGFP-N3 were mixed at 4, 16, 25, 37, or 42° C. for 30 minutes and transfected into Jurkat T cells for 30 minutes. After 24 hours, the expression changes of GFP were analyzed by immunoblotting, and the highest expression of GFP was found when the mixing temperature was 37° C. (FIG. 7). This indicates that the system of the present disclosure functions effectively under typical cell culture conditions or suitable temperature conditions for human application.

Thus, the temperature was set to 37° C., and 4 μM L₁₀-2×NLS and 2 μg pEGFP-N3 were mixed for different times (10, 20, 30, 40, 50, or 60 minutes). The expression changes of GFP were then analyzed using the same method as above, and it was found that GFP had the highest expression level when the mixing time was 30 minutes (FIG. 8). This shows the advantage that the desired transfection may be performed within a short period of time.

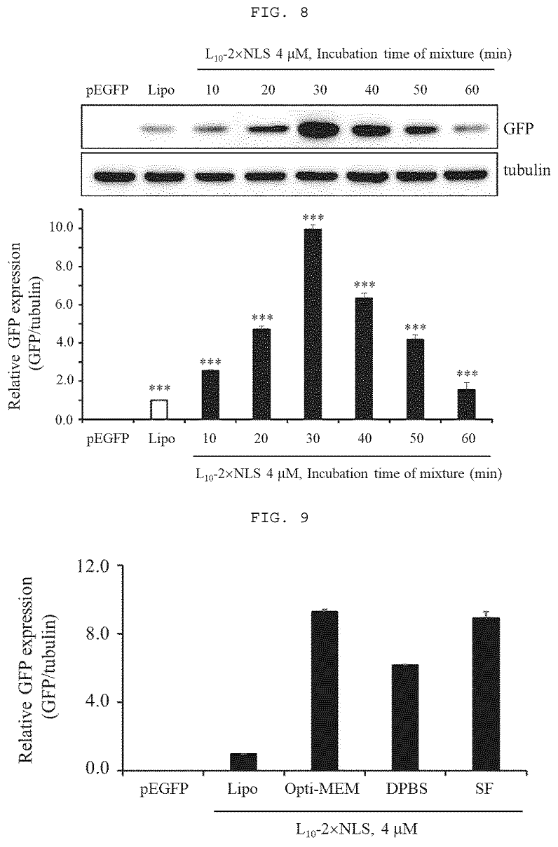

Finally, in an experiment as to the buffer used for the mixing, 4 μM of L₁₀-2×NLS and 2 μg of pEGFP-N3 were mixed with Opti-MEM, DPBS, or Serum free RPMI-1640 (SF) buffer, respectively. Then, the expression changes of GFP were analyzed using the same method as above, and it was confirmed that the transfection levels were maintained under different media conditions. This demonstrates that the system of the present disclosure is applicable and usable under various media conditions (FIG. 9).

Considering the above optimized temperature, time, and media conditions, the subsequent experiments were performed.

### 2-3. Plasmid DNA Delivery Effect Depending on Transfection Time

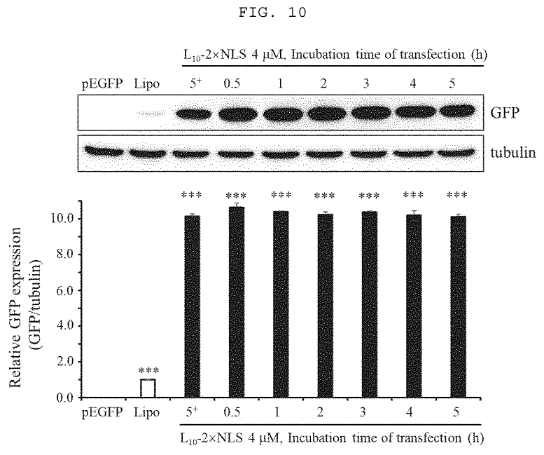

L₁₀-2×NLS (4 μM) and pEGFP-N3 (2 μg) were mixed according to the above optimized conditions and transfected into Jurkat T cells for 0.5, 1, 2, 3, 4, or 5 hours to compare the changes in GFP expression over time (FIG. 10).

The results showed that transfection for 0.5 or 1 hour was sufficient to deliver plasmid DNA.

### Example 3. Physicochemical Characterization of L₁₀-2×NLS and Plasmid DNA Complexes

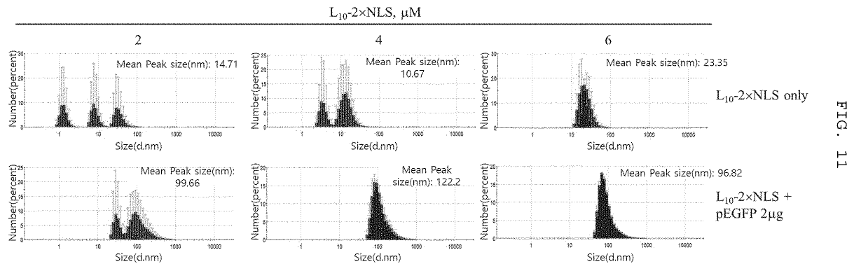

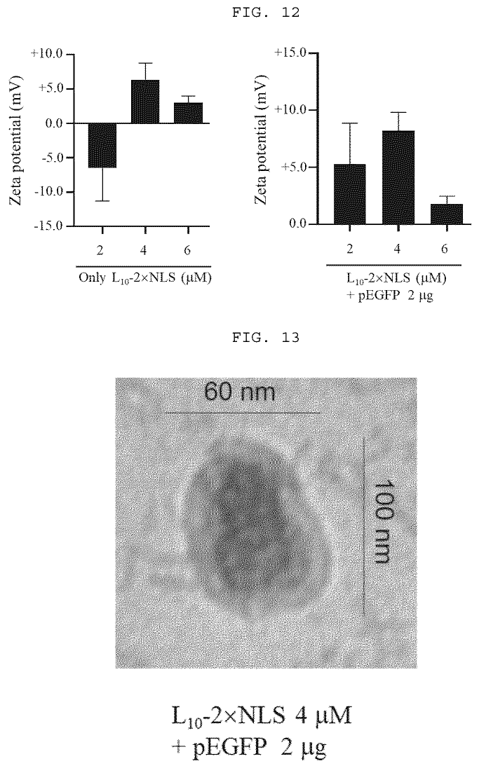

The size and zeta potential of the mixture of L₁₀-2×NLS alone (L₁₀-2×NLS only) and the mixture of L₁₀-2×NLS and pEGFP-N3 (L₁₀-2×NLS+pEGFP 2 μg) were analyzed using a particle size analyzer Zetasizer (Malvern Panalytical), respectively (FIGS. 11 and 12).

The results showed that the L₁₀-2×NLS and plasmid DNA complex was approximately 100 nm in size, and the zeta potential was positively charged between +2 and +6 mV, depending on the concentration of L₁₀-2×NLS.

Furthermore, the shape of the L₁₀-2×NLS and plasmid DNA complex was analyzed using a transmission electron microscope (TEM) (FIG. 13). As a result, the complex was found to be elliptical in shape with a size of 60-100 nm, as shown in FIG. 13.

This confirmed that L₁₀-2×NLS and plasmid DNA can form a nano-sized complex. It was also found that the polypeptide according to the present disclosure can form a sphere and act as a delivery material by loading nucleic acids within the sphere.

### Example 4. Effect of Transfection Using L₁₀-2×NLS on Cells

### 4.1. Confirmation of Damage and Death of Jurkat T Cells

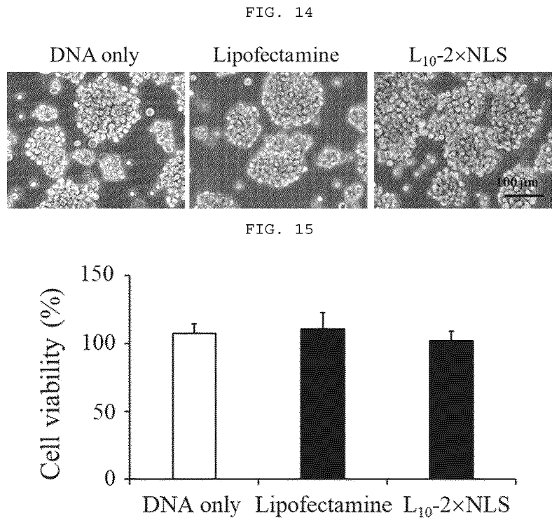

L₁₀-2×NLS (4 μM) and pEGFP-N3 (2 μg) were mixed and transfected into Jurkat T cells, and changes in cell morphology after 24 hours were observed (FIG. 14). The cell viability after transfection was analyzed using MTT assay (FIG. 15).

The results indicated that transfection of Jurkat T cells with L₁₀-2×NLS did not cause any damage or cell death.

### 4.2. Analysis of Delivery Efficiency and Location in Jurkat T Cells

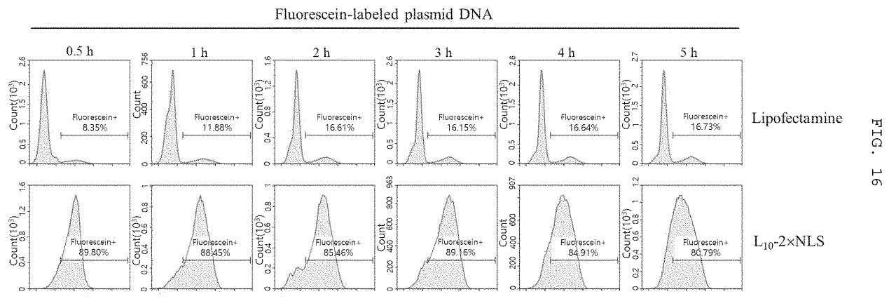

Fluorescein-labeled plasmid DNA (Takara) (2 μg) was mixed with L₁₀-2×NLS (4 μM) and transfected into Jurkat T cells for 0.5, 1, 2, 3, 4, or 5 hours. Then, the delivery efficiency of the plasmid DNA into Jurkat T cells was analyzed using Novocyte FACS (Agilent) (FIG. 16).

The results showed that lipofectamine was only 15% efficient even after 5 hours, while L₁₀-2×NLS delivered plasmid DNA to most cells with an efficiency of about 90% even after 0.5 hours.

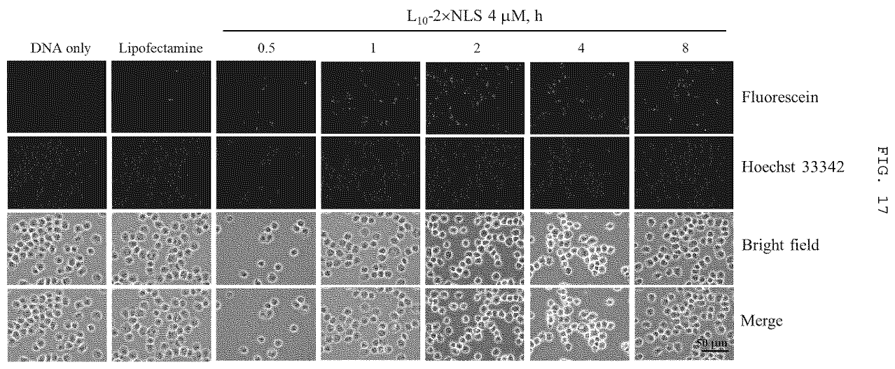

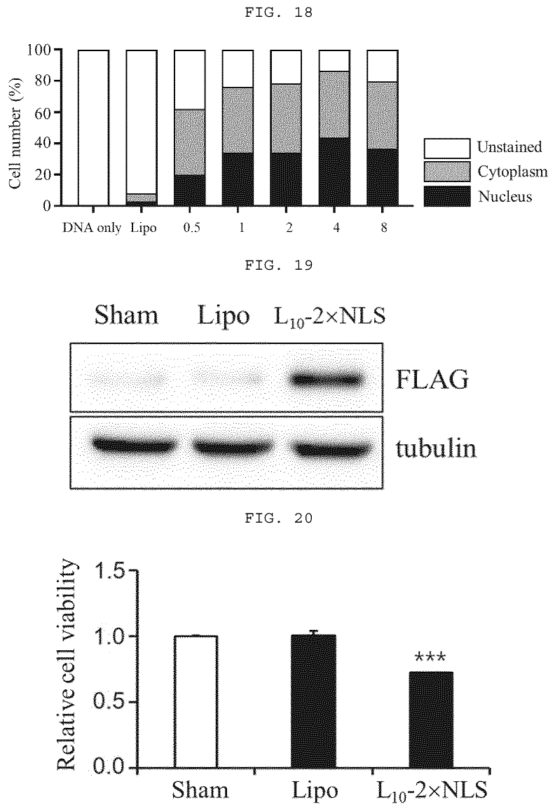

Next, fluorescein-labeled plasmid DNA and L₁₀-2×NLS were mixed and transfected into Jurkat T cells, followed by treatment with nucleus staining reagent Hoechst 33342 (ThermoFisher). The location of the plasmid DNA in Jurkat T cells was then confirmed using a fluorescence microscope (Leica) (FIG. 17), and the data were quantified and analyzed through graphical representation (FIG. 18). The results demonstrated that plasmid DNA was delivered to the majority of cells 1 hour after transfection with L₁₀-2×NLS, and was found in the nucleus of the cells at 4 hours, with an efficiency of approximately 40%.

This confirms that the transfection system according to the present disclosure can significantly increase the efficacy of transfection into cells compared to conventional transfection.

### Example 5. Intracellular Delivery of Plasmid DNA Using L₁₀-2×NLS and its Efficacy

### 5-1. Efficacy of CAR-T in Killing Blood Cancer Cells

L₁₀-2×NLS (4 μM) and FLAG-tagged CAR plasmid DNA (2 μg) were mixed and transfected into human primary T cells. After 24 hours, CAR expression in the human primary CD8+ T cells was confirmed by immunoblotting using FLAG antibody (FIG. 19).

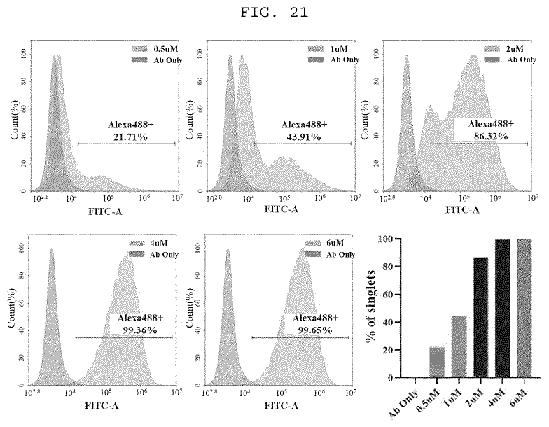

In order to determine whether CAR-T prepared according to the above method could kill blood cancer cells, CAR-T cells and blood cancer cells (Nalm6 cells) expressing luciferase were cultured together at a ratio of 10:1. After 6 hours, luciferase activity was measured, confirming that the CAR-T cells had a killing effect on blood cancer cells (FIG. 20).

The above results confirmed that the transfection system according to the present disclosure can be used to enhance cell killing efficacy by delivering therapeutic substances in a target-specific manner compared to conventional transfection.

### Example 6. Confirmation of Antibody Delivery Efficacy Using L₁₀-2×NLS

In order to confirm the possibility of delivery of not only nucleic acids but also other antibody drugs as a substance for delivery, an antibody was mixed with L₁₀-2×NLS and treated to confirm the change in transfection level.

### 6-1. Antibody Delivery Efficiency to Jurkat T Cells

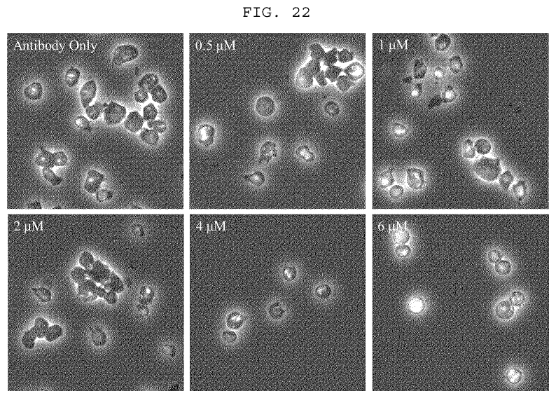

FITC-labeled IgG (Thermofisher) (1 μg) was mixed with L₁₀-2×NLS at different concentrations (0.5, 1, 2, 4, or 6 μM). Each mixture was treated with Jurkat T cells for 2 hours, and then the antibody delivery efficiency to cells was confirmed using Novocyte FACS (Agilent) (FIG. 21), and the presence of the corresponding antibodies in cells was observed using a fluorescence microscope (Leica) (FIG. 22).

As a result, the antibody was delivered into Jurkat T cells with an efficiency of approximately 90% when the concentration of L₁₀-2×NLS was 4 μM or higher.

In summary, it was demonstrated that L₁₀-2×NLS is usable to effectively deliver plasmid DNA or antibodies into cells.

This confirms that the desired polynucleotide, antibody, etc., can be delivered to various cells in a target-specific manner, and in particular, it is applicable to the development of immune cell therapeutics such as CAR-T cells.

1. A polypeptide comprising: 9, 10, or 11 consecutive leucines (Leu); and 1, 2, 3, or 4 repeats of peptide of SEQ ID NO: 1 linked thereto.

2. The polypeptide of claim 1, wherein the peptide of SEQ ID NO: 1 is linked to the C-terminal of leucine (Leu).

3. The polypeptide of claim 1, wherein the polypeptide comprises 10 or 11 consecutive leucines (Leu); and 2, 3, or 4 repeats of peptide of SEQ ID NO: 1 linked thereto.

4. The polypeptide of claim 1, wherein the polypeptide has any one sequence selected from the group consisting of SEQ ID NOS: 2 to 13.

5. The polypeptide of claim 4, wherein the polypeptide has the sequence of SEQ ID NO: 7, 8, 11 or 12.

6. A composition for intracellular transfection, comprising:
a polypeptide comprising 9, 10, or 11 consecutive leucines (Leu) and 1, 2, 3, or 4 repeats of peptide of SEQ ID NO: 1 linked thereto; and
a target substance.

7. The composition for intracellular transfection of claim 6, wherein the target substance is a compound, a protein, a nucleic acid or a drug.

8. The composition for intracellular transfection of claim 7, wherein the protein is at least one selected from the group consisting of an antibody, a receptor-bindable ligand peptide, a protein drug, a cytotoxic polypeptide, a cytotoxic protein, and a fluorescent protein.

9. The composition for intracellular transfection of claim 7, wherein the nucleic acid is selected from the group consisting of DNA, recombinant DNA, plasmid DNA, antisense oligonucleotide, aptamer, RNA, siRNA, shRNA, and miRNA.

10. The composition for intracellular transfection of claim 6, wherein the polypeptide is any one selected from the group consisting of SEQ ID NOS: 2 to 13.

11. The composition for intracellular transfection of claim 6, wherein the polypeptide forms a membrane to provide an outer layer with an inner compartment, thereby loading the target substance.

12. The composition for intracellular transfection of claim 6, wherein the cell is any one selected from the group consisting of a stem cell, a primary cell, an immune cell, a cancer cell, an epithelial cell, a skin cell, a gastrointestinal cell, a mucosal cell, and a lung cell.

13. (canceled)

14. (canceled)

15. (canceled)

16. (canceled)

17. (canceled)

18. (canceled)

19. (canceled)

20. (canceled)

21. (canceled)

22. The composition for intracellular transfection of claim 7, wherein the drug is any one selected from the group consisting of a compound drug, a bio drug, a nucleic acid drug, a peptide drug, a protein drug, a hormone, a contrast agent, and an antibody.

23. A method for delivering a target substance into a cell, comprising:
contacting the cell with a composition for intracellular transfection, comprising a polypeptide comprising 9, 10, or 11 consecutive leucines (Leu) and 1, 2, 3, or 4 repeats of peptide of SEQ ID NO: 1 linked thereto; and a target substance.

24. The method for delivering a target substance into a cell of claim 23, wherein the target substance is a compound, a protein, a nucleic acid, or a drug.

25. The method for delivering a target substance into a cell of claim 24, wherein the protein is at least one selected from the group consisting of an antibody, a receptor-bindable ligand peptide, a protein drug, a cytotoxic polypeptide, a cytotoxic protein, and a fluorescent protein.

26. The method for delivering a target substance into a cell of claim 24, wherein the nucleic acid is selected from the group consisting of DNA, recombinant DNA, plasmid DNA, antisense oligonucleotide, aptamer, RNA, siRNA, shRNA, and miRNA.

27. The method for delivering a target substance into a cell of claim 24, wherein the drug is any one selected from the group consisting of a compound drug, a bio drug, a nucleic acid drug, a peptide drug, a protein drug, a hormone, a contrast agent, and an antibody.

28. The method for delivering a target substance into a cell of claim 23, wherein the polypeptide is any one selected from the group consisting of SEQ ID NOS: 2 to 13.

29. The method for delivering a target substance into a cell of claim 23, wherein the cell is any one selected from the group consisting of a stem cell (e.g., an embryonic stem cell, a pluripotent stem cell, an induced pluripotent stem cell, a neural stem cell, a mesenchymal stem cell, a hematopoietic stem cell, a peripheral blood stem cell), a primary cell (e.g., a myoblast, a fibroblast), an immune cell (e.g., a NK cell, a T cell, a dendritic cell, an antigen presenting cell), a cancer cell, an epithelial cell, a skin cell, a gastrointestinal cell, a mucosal cell, or a lung cell.
